(19) United States
(12) Patent Application Publication
(10) Pub. No.: US 2011/0009621 A1
(43) Pub. Date: Jan. 13, 2011

(54) SUPPORTED TRANSITION METAL COMPLEX AND USE THEREOF IN CATALYSIS

(75) Inventors: Willi Bannwarth, Freiburg-im-Breisgau (DE); Florian Michalek, Freiburg-im-Breisgau (DE); Jurgen Ruhe, Eichstetten (DE)

Correspondence Address: VIKSNINS HARRIS & PADYS PLLP, P.O. BOX 111098, ST. PAUL, MN, 55111-1098, US

(73) Assignee: Raap Polymere GmbH, Tubingen (DE)

(21) Appl. No.: 12/095,999

(22) PCT Filed: Dec. 6, 2006
(86) PCT No.: PCT/US06/69365

§ 371 Date: Sep. 30, 2010

(30) Foreign Application Priority Data
Dec. 6, 2005 (DE) 10 2005 058 255.9
Dec. 9, 2005 (DE) 10 2005 058 980.4

(51) Int. Cl. C07D 223/08; B01J 31/12; C07C 67/303; C07C 41/20; C07D 207/46; C07D 211/94

(52) U.S. Cl. 540/604; 502/155; 560/122; 568/667; 548/542; 546/248

(57) ABSTRACT

The invention relates, in particular, to supported transition metal complexes based on a polystyrene matrix comprising structural units of the formula (Ia):

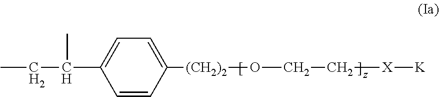

or supported transition metal complexes based on a silica gel matrix comprising structural units of the formula (II):

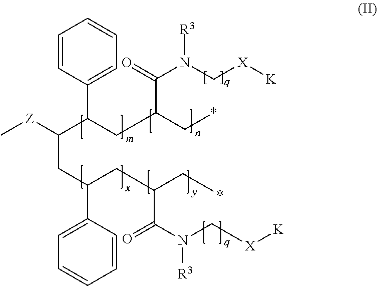

where the radicals and indices have the meanings given in the description.

The invention also relates to the use of the supported transition metal complexes in catalysis and corresponding processes for the transition metal-catalyzed conversion of starting material(s) into product(s). In particular, the invention relates to the field of olefin metathesis.

The present invention relates to supported transition metal complexes and their use in catalysis and also corresponding processes for the transition metal-catalyzed conversion of starting material(s) into product(s). In particular, the invention relates to the field of olefin metathesis.

The ability to separate off and reuse catalysts in a simple manner are of great importance both for industrial processes and for academic studies. From an economic point of view, a catalyst should ideally be completely reusable. In this respect, immobilized and heterogeneous catalysts have practical and economic advantages over homogeneous catalysts.

However, the selection of a suitable support and the type of bonding of the catalyst to the support is associated with some difficulties. In particular, care has to be taken to ensure that the high activities, selectivities and reaction rates which can be achieved using homogeneous transition metal catalysts are maintained and the products are as free as possible of metal and other impurities.

In the field of olefin metathesis, for example, initial attempts to bind catalysts based on ruthenium alkylidenes of the Grubbs type to polystyrene had only limited success and the resulting polymer-bonded catalyst was about two orders of magnitude less active than the homogeneous analogue. Furthermore, the work-up and reuse led to decreases in activity. There have now been further attempts to apply said metathesis catalysts to supports. An overview of these may be found, for example, in M. R. Buchmeiser, New J. Chem. 2004, 28, 549-557.

The use of supercritical carbon dioxide as reaction medium for transition metal-catalyzed reactions brings numerous advantages. Process engineering aspects such as nonflammability, ecological and toxicological acceptability, the in-principle avoidance of a gas/liquid phase boundary and possible simplifications in the work-up make the use of supercritical carbon dioxide attractive as an alternative to conventional solvents. Thus, it has been stated for the example of olefin metathesis that it is not only possible to “transfer” known reactions into the supercritical medium but the advantageous property profile of carbon dioxide allows the range of uses of an established reaction to be appreciably broadened (A. Fürstner, D. Koch, K. Langemann, W. Leitner, C. Six, Angew. Chem. 1997, 109, 2562-2565; Angew. Chem. Int. Ed. Engl. 1997, 36, 2466-1469; A. Fürstner. L. Ackermann, K. Beck, H. Hori, D. Koch, K. Langemann, M. Liebl, C. Six, W. Leitner, J. Am. Chem. Soc. 2001, 123, 9000-9006).

However, homogeneous catalysis in supercritical carbon dioxide is often adversely affected by the low solubility of the catalyst in the nonpolar solvent. This is at present frequently circumvented by modifying the ligand sphere of the catalyst by means of perfluorinated groups in order to increase the solubility. An example is the Stille coupling in supercritical carbon dioxide using Pd complexes whose phosphine ligands have perfluorinated substituents (T. Osswald, S. Schneider, S. Wang, W. Bannwarth, Tetrahedron Lett. 2001, 42, 2965-2967). While the introduction of perfluorinated substituents contributes to an increased solubility of the catalyst in supercritical carbon dioxide, it can also lead to increased leaching of the catalyst.

Proceeding from this prior art, it was an object of the present invention to provide catalysts whose use in supercritical carbon dioxide produces high conversions together with comparatively little leaching.

It has now surprisingly been found that the above object is achieved by bonding transition metal complexes to specific supports.

The present invention accordingly provides the supported transition metal complexes disclosed below and, in particular, in the claims.

The present invention also provides for the use of the supported transition metal complexes of the invention in catalysis.

Furthermore, a process for the transition metal-catalyzed conversion of starting material(s) into product(s) in supercritical carbon dioxide, which is characterized in that the catalyst is a supported transition metal complex, is disclosed.

Suitable supports are organic and inorganic materials which are essentially insoluble in supercritical carbon dioxide. However, the organic supports in particular can be swellable in supercritical carbon dioxide.

In an embodiment of the present invention, the supported transition metal complex is based on a polymer matrix which bears ethylene glycol oligomers for attachment of the transition metal complex. The oligomers generally comprise from 3 to 20, preferably from 5 to 15, for example 5, 6, 7, 8, 9 or 10, ethylene glycol units. Such ethylene glycol oligomers can be bound to polymer matrices in a manner known per se and can also be provided with suitable functional groups for attachment of the transition metal complexes. Accordingly, particular supported transition metal complexes of this type have structural units of the formula (I):

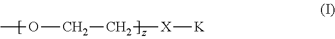

where
z is from 3 to 20;
X is a direct bond, oxygen, sulfur, —N(R¹)—, —C(═O)O—, —O(O═)C—, —N(R¹)(O═)C—, —C(═O)N(R¹)—, —O—CHR¹—O—, —OC(═O)N(R¹)—, —N(R¹)C(═O)O—, >C(═O) or >C(═S); and
K is a transition metal complex.

The way in which the transition metal complex is attached via the group X depends on the type of complex and in particular the functional groups provided by it for the purposes of bonding. In general, preference will be given to ether, ester, amide or urethane bonds because of the ease with which they can be synthesized, as long as these ensure stable attachment of the transition metal complex under the use conditions. The group X in the above formula (I) will therefore generally be oxygen, —C(═O)O—, —O(O═)C—, —N(R¹)(O═)C—, —C(═O)N(R¹)—, —OC(═O)N(R¹)— or —N(R¹)C(═O)O—. The same applies to the formula (Ia) and to the supported transition metal complexes of the formulae (II) and (IIa).

Many polymers are in principle available as polymer matrix and among these a large number have already been employed as support for transition metal complexes. These include, for example, polystyrenes, polyacrylamides, polyesters and polyurethanes, to name only a few. Among polystyrenes in particular, there are suitable polymer matrices which are also referred to as Merrifield resins, among which particular mention may be made of the polystyrenes crosslinked by means of, for example, divinylbenzene. Polystyrenes having a relatively low degree of crosslinking, for example those which can be obtained by crosslinking with from 0.5 to 2%, preferably about 1%, of divinylbenzene (in mol % based on the monomers used in the polymerization) are of particular importance for the purposes of the invention.

In a preferred embodiment of the present invention, the supported transition metal complex has a polystyrene matrix comprising structural units of the formula (Ia):

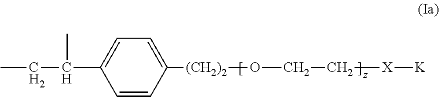

where
z is from 3 to 20;
X is a direct bond, oxygen, sulfur, —N(R¹)—, —C(═O)O—, —O(O═)C—, —N(R¹)(O═)C—, —C(═O)N(R¹)—, —O—CHR¹—O—, —OC(═O)N(R¹)—, —N(R¹)C(═O)O—, >C(═O) or >C(═S);
R¹ is hydrogen or C₁-C₄-alkyl; and
K is a transition metal complex.

In accordance with what has been said above, the index z in the formula (I) or (Ia) which indicates the average number of polyethylene units per polyethylene oligomer is preferably in the range from 5 to 15. According to the invention, particular preference is given to supported transition metal complexes of the formula (I) or (Ia) in which z is about 10.

The inventive polymer matrices having ethylene glycol oligomers bound thereto, for example polystyrene matrices comprising structural units of the formula (VI):

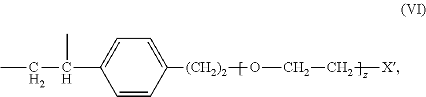

where X′ is a group which on reaction with an appropriately modified transition metal complex forms the group X, e.g. OH, SH or NH₂, can be obtained, for example, by reaction of hydroxyethyl-functionalized polystyrene with ethylene oxide and, if required, conversion of the terminal hydroxy groups into the groups X′ and can also be procured commercially, for example as the products marketed under the trade name HypoGel® from Rapp Polymere GmbH, Tübingen (Germany).

In a further embodiment of the invention, the supported transition metal complex is based on an inorganic matrix to which acrylamide-styrene copolymer is bound. Here, the acrylamide units perform the task of forming a bond to the matrix and also attaching the transition metal complex. Accordingly, particular supported transition metal complexes of this type have structural units of the formula (II):

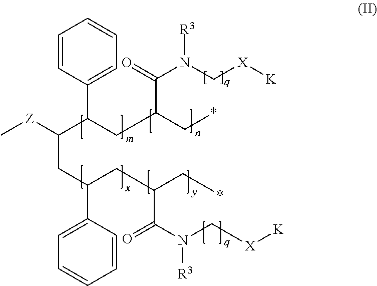

where
Z is a bond or a spacer;
m is from 1 to 5000;
n is from 1 to 5000;
x is from 1 to 5000;
y is from 1 to 5000;
X is a direct bond, oxygen, sulfur, —N(R¹)—, —C(═O)O—, —O(O═)C—, —N(R¹)(O═)C—, —C(═O)N(R¹)—, —OC(═O)N(R¹)—, —N(R¹)C(═O)O—, >C(═O) or >C(═S);
R¹ is hydrogen or C₁-C₄-alkyl;
R³ is hydrogen or C₁-C₄-alkyl;
q is from 2 to 5,
K is a transition metal complex,
where the monomer units indexed by m, n, x and y are randomly distributed.

The molar ratio of (m+x):(n+y), i.e. the molar ratio of styrene units to acrylamide units, is preferably in the range from 99:1 to 60:40, in particular in the range from 98:2 to 70:30 and particularly preferably in the range from 97:3 to 75:25.

As inorganic matrix, it is possible to use many inorganic materials which generally have hydroxy groups on their surface, e.g. silica gel, γ-Al₂O₃, molecular sieves (zeolites) and glass. Among the inorganic materials, silica gel is the most frequently used matrix since it is neutral and its properties and the possibility of modifying its surface have been well studied. Thus, the surfaces of such inorganic materials can be provided in a manner known per se with functional groups via which the copolymer can be bound. Silanes, in particular alkoxy silanes, which firstly bond to free hydroxy groups on the surface and secondly bear a function via which the copolymer can be bound, either directly or indirectly, to the surface of the inorganic matrix are generally used for this purpose. Sol-gel processes, in particular, which are known per se make it possible to provide tailored polysiloxane-comprising matrices having suitable functions.

In a preferred embodiment of the present invention, the supported transition metal complex has a silica gel matrix comprising structural units of the formula (IIa):

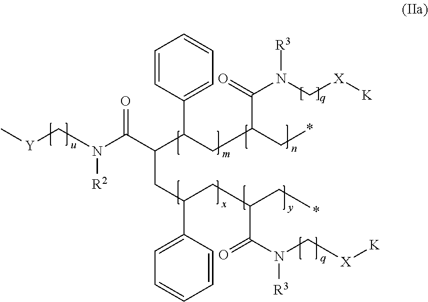

where
Y is Si, Si(OR⁴) or Si(OR⁴)₂, where the free valences of the silicon are bound to the alkylene (CH₂)ᵤ and also via oxygen to the silica gel;
m is from 1 to 5000;
n is from 1 to 5000;
x is from 1 to 5000;
y is from 1 to 5000;
X is a direct bond, oxygen, sulfur, —N(R¹)—, —C(═O)O—, —O(O═)C—, —N(R¹)(O═)C—, —C(═O)N(R¹)—, —O—CHR¹—O—, —OC(═O)N(R¹)—, —N(R¹)C(═O)O—, >C(═O) or >C(═S);
R¹ is hydrogen or C₁-C₄-alkyl;
R² is hydrogen or C₁-C₄-alkyl;
R³ is hydrogen or C₁-C₄-alkyl;
R⁴ is hydrogen or C₁-Cₐ-alkyl;
q is from 2 to 5,
u is from 2 to 5 and
K is a transition metal complex,
where the monomer units indexed by m, n, x and y are randomly distributed.

The molar ratio of styrene units to acrylamide units is preferably as indicated above for formula II.

The inventive inorganic matrices having acrylamide-styrene copolymer bound thereto, referred to as inorganic matrices, which comprise structural units of the formula (VII):

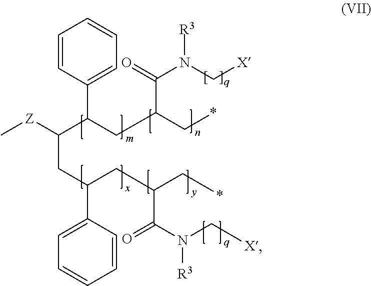

where X′ is a group which on reaction with an appropriately modified transition metal complex forms the group X, e.g. OH, SH or NH₂, can be obtained by means of the following process steps:
i) Introduction of a group which can be copolymerized with styrene and/or an acrylamide derivative of the formula (IX) into the inorganic matrix, where this group can be bound either directly or via a spacer to the inorganic matrix. A suitable group bound directly to the matrix is the vinyl group. Copolymerizable groups bound via a spacer include an allyl group, hydroxy-C₁-C₄-alkyl acrylate, hydroxy-C₁-C₄-alkyl methacrylate and in particular the group of the formula (VIII):

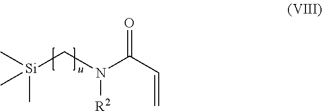

where R² and u are as defined above. The introduction of the latter group can be carried out in a customary way, for example by reaction of the matrix, in particular silica gel, with (R₄O)₃Si—(CH₂)ᵤ—NHR² or (R₄O)₃Si—(CH₂)ᵤ—OH. Here, the silane radical bonds to 1, 2 or 3 atoms of the matrix (oxygen atoms in the case of silica gel). The matrix which has been modified in this way is then reacted, for example, with acryloyl chloride or methacryloyl chloride or with a C₁-C₄-alkyl acrylate or methacrylate. However, (R₄O)₃Si-(CH₂)ᵤ—NHR² or (R₄O)₃Si-(CH₂)ᵤ—OH is preferably reacted with acryloyl chloride or methacryloyl chloride and the resulting amide or resulting ester is then bound to the matrix.
ii) Copolymerization of the modified matrix obtained according to (i) by means of styrene and an acrylamide derivative of the formula (IX):

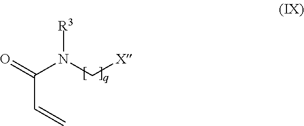

where X″ is protected or unprotected X′, e.g. N-acryloyl-N-methylpropylphthalimide (an acrylamide derivative of the formula (IX) in which R³ is methyl, q=3 and X″ is phthalimidyl). The relative proportions of the monomer units indexed by m, n, x and y are determined by the amounts used for the copolymerization and can also be adjusted by means of the polymerization conditions. The polymerization is advantageously carried out in an inert solvent such as toluene using a free-radical initiator which is soluble in the reaction mixture, for example an azo compound such as azoisobutyronitrile (AIBN);
iii) if required, conversion of the group X″ into the group X′, in particular by removing protection, for example by converting the phthalimide group into an amino group.

The transition metal complex which generally comprises one or more transition metals or transition metal ions and one or more complexing ligands is generally bound via a functional group which provides a generally covalent bond under the use conditions to the supports in a manner as described above either via the ethylene glycol oligomers or the styrene-acrylamide copolymers. Here, as stated above, the group X represents the point of attachment.

The range of transition metal complexes suitable for catalytic applications is generally not subject to any restriction. Only the stability of the complexes under the immobilization conditions, i.e. the reaction conditions for attachment of the transition metal complex to the support, has to be taken into account. Most transition metal complexes known from homogeneous catalysis can therefore be attached to supports in the above way by means of appropriately functionalized complexing ligands.

Typical transition metal complexes which are of interest here include a complexes, for example organocopper complexes, in particular those for catalyzing conjunctive additions onto O-unsaturated carbonyl compounds, palladium complexes, in particular those for catalyzing the Heck reaction, Stille coupling or Suzuki coupling, iron complexes, in particular Collman's reagent and iron-σ-allyl complexes, and titanium complexes, in particular those for catalyzing geminal dimethylations; carbene complexes, for example Fischer carbene complexes, in particular those for catalyzing ester/amide-analogous reactions or (2+2)-cycloadditions, and Schrock alkylidene complexes, in particular the Tebbe reagent or those for catalyzing carbonyl olefination with esters or olefin metathesis; alkene and alkyne complexes, for example Fe(CO)₄ complexes, in particular those for catalyzing nucleophilic additions, and alkyne-cobalt carbonyl complexes, in particular those for catalyzing Nicholas reactions or Pauson-Khand reactions, also those for catalyzing Wacker processes, intramolecular oxypalladations and also catalytic hydrogenations and hydrometalations, in particular Wilkinson catalysts, for example for catalyzing hydroformylations and hydrosilylations; π-allyl complexes, for example π-allyl complexes of palladium, in particular those for catalyzing cyclizations, pentanilations and Bäckvall oxidations; η⁴-diene complexes, for example Fe(CO)₃ complexes; η⁵-Fe(CO)₃ complexes; ferrocene and related complexes, in particular those for catalyzing oxidations, protonations, electrophilic substitutions and metalations; and η⁶-arene complexes, in particular those for catalyzing nucleophilic additions, nucleophilic substitutions and lithiations.

In a particular embodiment of the present invention, the supported transition metal complex is suitable as olefin metathesis catalyst. Such complexes include, in particular, tungsten-, molybdenum- and ruthenium-alkylidene complexes, among which the molybdenum and especially the ruthenium complexes are of particular importance according to the invention.

Said molybdenum-alkylidene complexes have, for example, a structure of the formula (III):

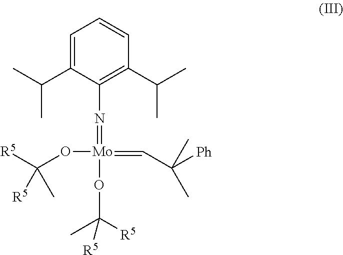

where
R⁵ is methyl or trifluoromethyl.
Such molybdenum-alkylidene complexes are generally referred to as Schrock catalysts.

Said ruthenium-alkylidene complexes have, for example, a structure of the formula (IVa):

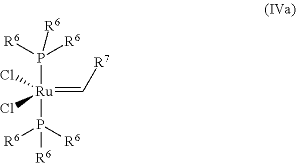

where
R⁶ is phenyl or cyclohexyl and
R⁷ is phenyl or CH═CPh₂.

Such ruthenium-alkylidene complexes are generally referred to as first generation Grubbs catalysts.

Further representatives of said ruthenium-alkylidene complexes have, for example, a structure of the formula (IVb):

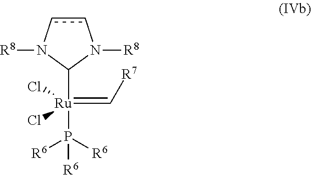

where
R⁶ is cyclohexyl;
R⁷ is phenyl and
R⁸ is 2,4,6-trimethylphenyl (mesityl),
where “---” indicates an optional double bond.

Such ruthenium-alkylidene complexes are generally referred to as second generation Grubbs catalysts.

Further representatives of said ruthenium-alkylidene complexes have, for example, a structure of the formula (IVc):

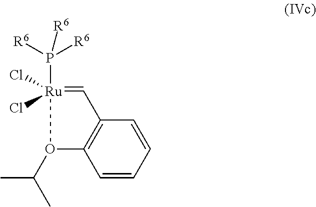

where
R⁶ is cyclohexyl.

Such ruthenium-alkylidene complexes are generally referred to as first generation Hoveyda catalysts.

Further representatives of said ruthenium-alkylidene complexes have the structure of the formula (IVd):

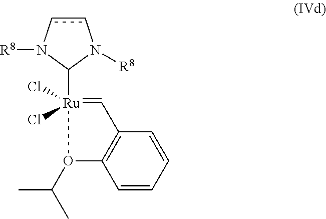

where
R⁸ is 2,4,6-trimethylphenyl (mesityl),
where “---” indicates an optional double bond.

Such ruthenium-alkylidene complexes are generally referred to as second generation Hoveyda catalysts. Supported transition metal complexes based on Hoveyda catalysts represent a particular embodiment of the present invention.

In the supported form according to the invention, said transition metal complexes are generally modified so that attachment to the support is ensured. The term “transition metal complex” as used here thus generally refers to a modified embodiment of the transition metal complexes known per se from homogeneous catalysis, for example the above-described ruthenium-alkylidene complexes. Thus, the structures of the transition metal complexes K in the above formulae are derived from or based on the structures of the transition metal complexes known per se from homogeneous catalysis, for example the structures of the formula (IVa), (IVb), (IVc) or (IVd).

There are many possible way of attaching such transition metal complexes to the supports according to the invention, in particular the modified matrices of the formulae (VI) and (VII) and their specific embodiments. In general, at least one of the transition metal ligands is provided with a functional group which allows bonding. This will be illustrated by way of example for the above-described ruthenium-alkylidene complexes. A distinction can be made in principle between the following methods:
i) replacement of the phosphine ligand;
ii) replacement of the alkylidene ligand;
iii) replacement of the N-heterocyclic carbene ligand; or
iv) replacement of a halogen ligand.

An example of method (i) is the replacement of at least one phosphine ligand, i.e., in particular, at least one group P(R⁶)₃ in the complexes of the formulae (IVa), (IVb) and (IVc), by corresponding phosphines in which at least one radical R⁶ is modified so that attachment can occur via these. In this way, it has been possible, for example, to bond ruthenium-alkylidene complexes of the above type to a polystyrene matrix crosslinked with 2% of divinylbenzene (S. T. Nguyen and R. H. Grubbs, J. Organomet. Chem. 1995, 497, 195-200) or to silica gel (K. Mehlis, D. De Vos, P. Jakobs and F. Verpoort, J. Mol. Catat A: Chem., 2001, 169, 47).

An example of method (ii) is replacement of the alkylidene ligand, in particular the ═CHR⁷ group in the above-described ruthenium-alkylidene complexes of the formulae (IVa) and (IVb), by alkylidene bound to a support. In this way, it has been possible, for example, to bond ruthenium-alkylidene complexes of the above type to poly(vinylstyrene-co-divinylbenzene) (M. Ahmed, A. G. M. Barrett, D. C. Braddock, S. M. Cramp, P. A. Procopiou, Tetrahedron Lett. 1999, 40, 8657-8662).

A variant of method (ii) which is preferred according to the invention is functionalization of the 2-isopropoxybenzylidene ligand in the ruthenium-alkylidene complexes of the formulae (IVc) and (IVd). Thus, in particular, suitable substituents can be introduced in the 5 position. These include carboxyalkyl groups, for example —CH₂—CH₂—COOH (cf. S. B. Garber, J. S. Kingsbury, B. L. Gray, A. H. Hoveyda, J. Am. Chem. Soc. 2000, 122, 8168-8179; J. S. Kingsbury, S. B. Garber, J. M. Giftos, B. L. Gray, M. M. Okamoto, R. A. Farrer, J. T. Fourkas, A.H. Hoveyda, Angew. Chem. 2001, 113, 4381-4386; Angew. Chem., Int. Ed. 2001, 40, 4251-4256; J. S. Kingsbury, A. H. Hoveyda, J. Am. Chem. Soc. 2005, 127, 4510-4517; S. J. Connon, S. Blechert, Bioorg. Med. Chem. Lett. 2002, 12, 1873-1876), carboxyalkenyl groups, for example —CH═CH—COOH (F. Koç, F. Michalek, L. Rumi, W. Bannwarth, R. Haag, Synthesis 2005, 3362-3372), a hydroxy group (Q. Yao, Angew. Chem. 2000, 112, 4060-6042; Angew. Chem. Int. Ed. 2000, 39, 3896-3898; S. Randl, N. Buschmann, S. J. Connon, S. Blechert, Synlett 2001, 1547-1550), hydroxyalkyl groups, for example hydroxymethyl (Q. Yao, A. R. Motta, Tetrahedron Lett. 2004, 45, 2447-2451) or halogen atoms, for example bromine (K. Grela, M. Tryznowski, M. Bieniek, Tetrahedron Lett. 2002, 43, 9055-9059).

A further possibility is to introduce a similar substituent in a position other than the 5 position of the 2-isopropoxybenzylidene ligand, for example a hydroxy group in the 3 position (C. Fischer, S. Blechert, Adv. Synth. Catal. 2005, 347, 1329-1332).

As an alternative to introduction of a substituent on the phenyl ring of benzylidene, it is possible to modify the isopropoxy substituent in the 2 position of the benzylidene ligand in the ruthenium-alkylidene complexes of the formulae (IVc) and (IVd). For example, the isopropoxy group can be replaced by a 1-carboxyhexan-2-oxy group (J. Dowden, J. Savovic, Chem. Commun. 2001, 37-38).

An example of method (iii) is introduction of a substituent in the 4-position of the 1,3-disubstituted imidazol-2-ylidene or 4,5-dihydroimidazol-2-ylidene ligand or replacement of one or both substituents R⁸ in the ruthenium-alkylidene complexes of the formulae (IVb) and (IVd) in such a way that attachment is possible via these. For example, hydroxyalkyl groups, for example hydroxymethyl, can be introduced in the 4 position of the ligand (S. Randl, N. Buschmann, S. J. Connon. S, Blechert, Synlett 2001, 1547-1550; S. C. Schürer, S. Gessler, N. Buschmann, S. Blechert, Angew. Chem. 2000, 112, 4062-4065; Angew. Chem. Int Ed. 2000, 39, 3898-3901) or a hydroxyalkyl group, for example hydroxyhexyl, can be bound to a nitrogen of the ligand (S. Prüths, C. W. Lehmann, A. Fürstner, Organometallics 2004, 23, 280-287).

An example of method (iv) is replacement of a halogen of the above-described ruthenium-alkylidene complexes by suitable, appropriately functionalized ligands such as perfluoroglutaric acid (J. O. Krause, S. Lubbad, O, Nuyken and M. R. Buchmeiser, Adv. Synth. Catal., 2003, 345, 996).

Transition metal complexes which have been modified in this way can be bound to the support (i.e. to the group X′ to form the group X) either directly or via a spacer.

The above descriptions of methods accordingly provide many possible ways of attaching transition metal complexes to the supports according to the invention.

According to the invention, particular preference is given to K in the supported transition metal complexes of the formulae (Ia) and (Ib) being a group of the formula (V):

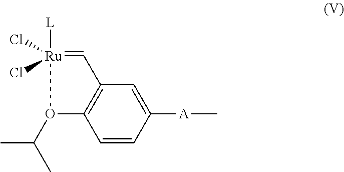

where
L is P(R⁶) or 1,3-substituted 4,5-dihydroimidazol-2-ylidene, where R⁶ is as defined above; and
A is a divalent radical which is bound to X and is preferably selected from among C₁-C₁₀-alkylene and C₂-C₁₀-alkenylene which may each be interrupted by 1, 2 or 3 heteroatoms which are preferably selected from among oxygen, nitrogen and sulfur.

In a particularly preferred embodiment, A is methylene, ethylene, propylene or ethenylene.

The supported transition metal complexes of the invention are, depending on the type of complex, employed in the catalysis of many chemical reactions. The use of the above-described supported ruthenium-alkylidene complexes in olefin metathesis is a particular use within the scope of the invention.

The process of the invention for the transition metal-catalyzed conversion of starting material(s) into product(s) in supercritical carbon dioxide is applicable in principle to any reactions which can be catalyzed by means of transition metal complexes in supercritical carbon dioxide. These include, in particular, the reactions which are mentioned above in relation to the transition metal complexes and are catalyzed by these. To avoid repetition, what has been said above is incorporated by reference at this point.

Olefin metathesis is of particular significance. This is, according to the invention, a transition metal-catalyzed reaction in which the alkylidene groups are formally exchanged between two substituted alkenes. It is thus a catalytic process for the breaking and reforming of C—C double bonds.

In the field of olefin metathesis, a distinction can be made between various embodiments according to the starting materials to be reacted and the products to be expected. The most important representatives include ring-opening metathesis polymerization (ROMP for short), acyclic diene metathesis (ADMET for short), cross metathesis (CM for short), ring-opening metathesis (ROM for short) and ring-closing metathesis (RCM for short). Further important metathesis reactions are 1-alkyne polymerization, enyne metathesis, ring-opening cross metathesis and tandem metathesis, for example tandem ring-opening ring-closing metathesis and combined ring-opening ring-closing cross metathesis.

In a particular embodiment, the present invention provides a process for transition metal-catalyzed ring-closing metathesis. This is, in particular, an intramolecular reaction of α,ω-diolefins to form corresponding cyclic products. α,ω-Diolefins are compounds which have two terminal double bonds.

The supported transition metal complexes of the invention make it possible for a major part of the starting material (or starting materials) introduced to be reacted in a short time, in particular in supercritical carbon dioxide. Optimization of the reaction conditions, for example the reaction temperature, the carbon dioxide pressure, the reaction time and the amounts of starting material and supported transition metal complex to be used, is carried out by a person skilled in the art. For example, α,ω-diolefins can be converted in a ring-closing metathesis into corresponding cyclic products in a conversion of more than 80% at a temperature in the range from 20 to 60° C., in particular from 30 to 50° C., for example about 40° C., a carbon dioxide pressure in the range from 80 to 160 bar, for example about 150 bar, a concentration of supported transition metal complex in the range from 1 to 5 mol %, preferably from 2 to 3 mol %, for example about 2.5 mol % (Ru based on the amount of starting material), in a few hours, for example from 10 to 24 hours.

Suitable apparatuses for carrying out reactions in supercritical carbon dioxide are known to those skilled in the art, as is the establishment of the supercritical state.

The products of value resulting from the reaction can be isolated in a manner known per se.

In the above formulae, C₁-C₄-alkyl is methyl, ethyl, n-propyl, isopropyl, n-butyl, sec-butyl, i-butyl or t-butyl. Preference is given to ethyl and in particular methyl.

The following examples illustrate the invention without restricting its scope.

### EXAMPLES

### Example 1

### Preparation of the Supported Transition Metal Complex

1: Preparation of trans-3-(4-isopropoxy-3-vinylphenyl)acrylic acid

trans-3-(4-Isopropoxy-3-vinylphenyl)acrylic acid was prepared by reacting 5-bromo-2-hydroxybenzaldehyde with 2-iodopropane in DMF in the presence of K₂CO₃ and Cs₂CO₃ to form 5-bromo-2-isopropoxybenzaldehyde, then reacting this with ethyl acrylate in anhydrous DMF (dimethylformamide) in the presence of Pd(OAc)₂ and P(o-Tol)₃ and also triethylamine to form trans-3-(3-formyl-4-isopropoxyphenyl)ethyl acrylate, reacting this with methyltriphenylphosphonium bromide and BuLi in anhydrous THF (tetrahydrofuran) to form trans-3-(4-isopropoxy-3-vinylphenyl)ethyl acrylate and finally reacting this in 1,4-dioxane with an aqueous KOH solution to form trans-3-(4-isopropoxy-3-vinylphenyl)acrylic acid. This reaction sequence is described in more detail in F. Koç, F. Michalek, L. Rumi, W. Bannwarth, R. Haag, Synthesis 2005, 3362-3372.

2: Coupling of trans-3-(4-isopropoxy-3-vinylphenyl)acrylic acid to amino groups of various supports

trans-3-(4-Isopropoxy-3-vinylphenyl)acrylic acid is coupled to protected or unprotected amino groups of the support concerned by dissolving the acrylic acid together with HOBt in DMF, adding DCC (dicyclohexylcarbodiimide) and Huenig's base to the solution and adding the resulting coupling mixture to the support suspended in fresh DMF. As soon as free amino groups could not longer be detected, the solid phase was reacted with triethylamine and DMAP in dichloromethane and an acetic anhydride solution.

This reaction is described in more detail in F. Koç, F. Michalek, L. Rumi, W. Bannwarth, R. Haag, Synthesis 2005, 3362-3372.

In this way, trans-3-(4-isopropoxy-3-vinylphenyl)acrylic acid was coupled to the following supports:

### a) HypoGel® 400

HypoGel® 400 is the trade name for a hydrophilic resin which is marketed by Rapp Polymere, Tübingen (Germany) and is based on a polystyrene matrix which has a low degree of branching (1% of divinylbenzene) and comprises structural units of the formula (VIa):

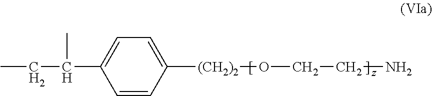

where z is 10.
b) Hybrid silica gel

The term hybrid silica gel refers to a support which is based on silica gel and has a coating of acrylamide-styrene copolymer. The acrylamide units are alkylated, in each case with a methyl group and a propylene group which either has a free amino group or is bound via alkoxysilane groups to the silica gel. The support accordingly has a silica gel matrix comprising structural units of the formula (VII):

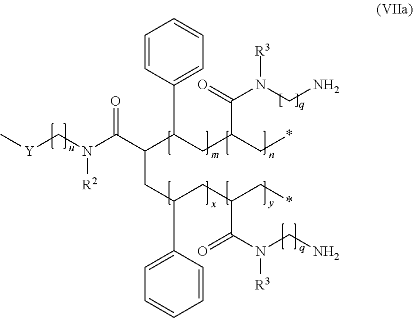

where
M⁴ is silica gel;
Y is Si, Si(OR⁴) or Si(OR⁴)₂, where the free valences of the silicon are bound to the alkylene (CH₂)ᵤ and also via oxygen to the silica gel;
m is from 1 to 5000;
n is from 1 to 5000;
x is from 1 to 5000;
y is from 1 to 5000; and
R¹, R² and R³ are each methyl;
R⁴ is methyl; and
u and q are each 3.

Hybrid silica gel is therefore a support which has a comparatively rigid core structure which is coated with an ultrathin layer (in the nanometer range) of an acrylamide-styrene copolymer.

The preparation of hybrid silica gel is illustrated below:

The supports based on silicon dioxide which were employed were suspended in toluene under dry nitrogen. The silane monomer unit and triethylamine, dissolved in toluene, were subsequently added, heated to commencement of reflux (120° C.) and agitated by means of a shaking apparatus for 3 hours. The experiments carried out are shown in table 1 below:

TABLE 1
Formulations

| | | Amount of | Volume of | Amount of |
|---|---|---|---|---|
| Modified | mmol of | support | toluene | triethylamine |
| support¹⁾ | silane | (g) | (ml) | (mmol) |
| LC700-ME | 8.4 | 6 | 200 | 54 |
| GB80-ME | 4.2 | 10 | 125 | 36 |
| GB80-AA | 4.2 | 10 | 80 | 28 |
| GB250-AA | 2.5 | 5 | 60 | 21 |

¹⁾LC 700 (Lichrosphere) is a commercial silica gel, GB 80 and 250 are glass beads of different diameters. The suffix ME means that the support has been modified using (MeO₃)Si(CH₂)₃OH and methacryloyl chloride (in the case of the suffix AE, acryloyl chloride was used instead of methacryloyl chloride). The suffix AA means that the support was modified using (MeO₃)Si(CH₂)₃NHCH₃ and acryloyl chloride.

The modified supports based on silica gel were centrifuged at 12 500 rpm (the modified glass beads settled quickly enough for no centrifugation to be necessary) and washed with toluene, ethanol, ethanol/water (1/1, v/v, acidified with HCl), ethanol/water (1/1, v/v), ethanol and diethyl ether to remove excess triethylamine. The colorless products obtained were dried for 18 hours at 0.01 mbar.

Copolymerization to form matrix-bonded poly(styrene-co-N-acryloyl-N-methylpropyl-phthalimide), PS-AC3pht

The modified supports were admixed with toluene and styrene (main monomer) in the amounts indicated in table 2 in a Schlenk tube. N-Acryloyl-N-methylpropylphthalimide (functionalizing monomer) and AIBN were subsequently dissolved in the liquid phase. The solution was degassed under reduced pressure by means of 5 freeze-thaw cycles and the mixture was thermostatted to 60.0° C. After some time, the polymerization was stopped by introduction of air and cooling. The desired products were separated off by centrifugation or allowing them to settle. The polymerization conditions are shown in table 2.

TABLE 2
Copolymerizations

| | Amount | Amount | Conc. | Vol. | Vol. | Amount | Conc. | |
|---|---|---|---|---|---|---|---|---|
| Modified | of support | of fM | of fM | of M | of solvent | of I | of I | t |
| support | (mg) | (mg) | (mol %) | (ml) | (ml) | (mg) | (mmol · l⁻¹) | (h) |
| LC700-ME | 240 | 946 | 5 | 8.0 | 16 | 36 | 9 | 24 |
| LC700-ME | 5000 | 1320 | 10 | 5.0 | 10^{#} | 22 | 9 | 30 |
| GB80-ME | 2000 | 1426 | 5 | 12.0 | 24 | 18 | 3 | 24 |
| GB80-ME | 2000 | 2836 | 10 | 10.8 | 24 | 17 | 3 | 25 |
| GB80-ME | 2000 | 5673 | 20 | 9.6 | 24 | 17 | 3 | 25 |
| GB80-AA | 13500 | 3160 | 10 | 12.0 | 100 | 80 | 5 | 30 |
| GB250-AA | 5000 | 2370 | 20 | 4.0 | 8^{#} | 18 | 9 | 26 |

In the table: fM = functionalized monomer; M = main monomer; I = initiator
Matrix-bonded poly(styrene-co-N-acryloyl-N-methylpropylamine), PS-AC3amine

The copolymers of the preceding step were covered with THF and admixed with hydrazine hydrate (up to a 50-fold excess). The mixture was shaken at 60° C. and 180 rpm for 18 hours and subsequently filtered, and the solids which remained were washed with toluene, dichloromethane and toluene again. This was followed by freeze drying from benzene.

To quantify the number of amino groups, a DMT dye having disulfide linkers was coupled to the amino functions. Determination of the sulfur content and splitting-off of the dye for the UV determination indicated the amount of available amino groups; see table 3 below.

In the case of the glass beads, the splitting-off of the phthalimide was carried out using methylamine. A suspension of 1.9 g of the phthalimide and 20 ml of a 2M methylamine solution in THF was heated to 60° C. and shaken at 160 rpm for 18 hours. The glass beads settled on cooling and were washed 6 times with 40 ml each time of THF and twice with 40 ml each time of diethyl ether and dried under reduced pressure for 8 hours.

TABLE 3
Analytical results

| | C* | H* | N* | m′_{poly}^{#} | δ(NH₂)° |
|---|---|---|---|---|---|
| Product | (%) | (%) | (%) | (g) | (μmol/g) |
| LC700-ME-PS- | 25.60 | 3.43 | 0.53 | 0.350 | 20 |
| AC₃amine(10%) | | | | | |
| GB80-ME-PS- | 11.00 | 0.86 | 0.22 | 0.115 | — |
| AC₃amine(5%) | | | | | |
| GB80-ME-PS- | 11.17 | 0.86 | 0.33 | 0.120 | 90 |
| AC₃amine(10%) | | | | | |
| GB80-ME-PS- | 11.84 | 1.14 | 0.66 | 0.139 | 90 |
| AC₃amine(20%) | | | | | |
| GB80-AA-PS- | 7.83 | 1.19 | 0.60 | 0.081 | 60-80 |
| AC₃amine(10%) | | | | | |
| GB250-AA-PS- | 19.46 | 2.51 | 1.62 | 0.242 | 150 |
| AC₃amine(20%) | | | | | |

*obtained by elemental analysis
^{#}per g of SiO₂, determined from the elemental analysis
°measurements of the coupled DMT disulfide units

4: Loading with ruthenium

The respective supports having trans-3-(4-isopropoxyvinylphenyl)acrylic acid bound thereto was suspended in anhydrous dichloromethane under an argon atmosphere and either (PCy₃)₂Cl₂Ru=CHPh or (4,5-H₂lMES)(PCy₃)Cl₂Ru=CHPh (Cy=cyclohexyl; IMES=1,3-bis(2,5,6-trimethylphenyl)imidazol-2-ylidene) and CuCl were added thereto.

This reaction is described in more detail in F. Koç, F. Michalek, L. Rumi, W. Bannwarth, R. Haag, Synthesis 2005, 3362-3372.

To remove insoluble Cu-phosphine residues, the following procedure was employed:

In the case of transition metal complexes supported on hybrid silica gel, the reaction mixture was filtered and the filtrate was washed until it was colorless. Neocuproin was subsequently added to remove Cu ions selectively and their absence was confirmed by means of XPS measurements.

In the case of transition metal complexes supported on HypoGel® 400, the procedure described in S. J. Connon, S. Blechert, Bioorg. Med. Chem. Lett. 2002, 12, 1873-1876, was employed.

The Ru loading of all the supported transition metal complexes was subsequently determined by means of ICP-MS (in the case of transition metal complexes supported on hybrid silica gel) or by means of AAS (in the case of transition metal complexes supported on HypoGel® 400). The results are shown in table 4 below:

TABLE 4

| Support | Ligand | Loading [μmol/g] |
|---|---|---|
| hybrid silica gel | PCy₃ | 32 |
| | H₂IMES | 56 |
| HypoGel ® 400 | PCy₃ | 67 |
| | H₂IMES | 217 |

### Example 2

### Catalysis

The reactions were carried out in a steel autoclave from NWA GmbH, Lörrach (Germany) (variable volume of 29-61 ml). The autoclave was equipped with a sapphire window and an internal stirrer. A pressure module having a maximum output of 600 bar was used for introducing carbon dioxide.

The general procedure for carrying out the catalytic reaction under supercritical conditions was as follows: the supported transition metal complex (2.5 mol %) was introduced into a specially designed small glass vessel in the autoclave. The reactor was carefully pressurized with CO₂ to 100 bar at 40° C. The substrate was then introduced via a loop connected to the autoclave and the pressure was increased to 140 bar. After 24 hours, the reactor was vented at 40° C. The organic compounds were collected in a flask containing dichloromethane and ethyl vinyl ether. The conversion was then determined by means of ¹H-NMR. All products were analyzed by means of ¹H-NMR and mass spectrometry and compared to the literature data.

The catalytic properties of the supported transition metal complexes were assessed by means of the conversion of N,N-diallyltosylamide into N-tosylpyrroline by ring-closing metathesis.

Table 5 below shows the conversions for each of the supported transition metal complexes used:

TABLE 5

| Support | Ligand | Conversion (cycle 1, 2, 3, 4, 5) [%] |
|---|---|---|
| hybrid silica gel | PCy₃ | 83, 75, 66, 66, 56 |
| | H₂IMES | 93, 92, 77, 54, 57 |
| HypoGel ® 400 | PCy₃ | 95, 95, 90, 89, 84 |
| | H₂IMES | 92, 95, 85, 34, 12 |

For comparison, the same reaction was carried out using the corresponding unsupported transition metal complexes (PCy₃)₂Cl₂Ru=CHPh and (H₂IMES)(PCy₃)Cl₂Ru=CHPh. The conversions achieved are shown in table 6 below:

TABLE 6

| Transition metal complex | Conversion (cycle 1, 2, 3, 4, 5) [%] |
|---|---|
| (PCy₃)₂Cl₂Ru | >98, >98, >98, >98, >98 |
| (H₂IMES)(PCy₃)Cl₂Ru | >98, >98, >98, >98, 90 |

The comparative transition metal complexes (PCy₃)₂Cl₂Ru and (H₂IMES)(PCy₃)Cl₂Ru each gave quantitative conversion of the starting material. 5 successive cycles without loss of activity were possible.

Although not complete, the supported transition metal complexes displayed similarly good conversions.

Further studies using (H₂IMES)(PCy₃)Cl₂Ru=CHPh supported on hybrid silica gel showed that the reaction was independent of pressure in the range from 80 to 160 bar of carbon dioxide.

Furthermore, studies on the time dependence showed that the reaction was complete after only one hour when using the comparative transition metal complexes, while the conversions at this point in time were only 50% when the transition metal complex supported on hybrid silica gel was used (a 90% conversion was achieved after about 6 hours).

The Ru content of the product (N-tosylpyrroline) determined by means of ICP-AES was surprisingly only 18 or 21 ppm when using the two transition metal complexes supported on hybrid silica gel, while the product was contaminated with 100 ppm when using the comparative transition metal complexes. A content of only about 20 ppm of ruthenium at a conversion of over 90% is remarkable.

Further starting materials and associated ring-closing metathesis products together with the conversion which can be achieved using (H₁IMES)(PCy₃)Cl₂Ru=CHPh supported on hybrid silica gel are shown in table 7 below.

TABLE 7

| Starting material | Product | Conversion [%] |
|---|---|---|
| 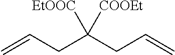 | 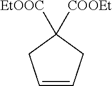 | 74 |
| 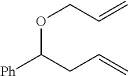 | 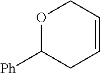 | 90 |
| 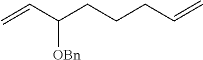 | 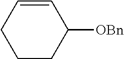 | > 98 |
| 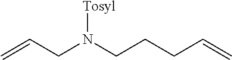 | 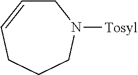 | > 98 |
| 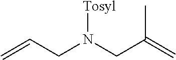 | 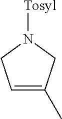 | 92 |
| 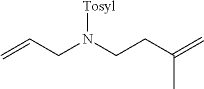 | 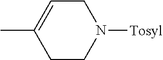 | 93 |

1. A supported transition metal complex based on a polystyrene matrix comprising structural units of the formula (Ia): 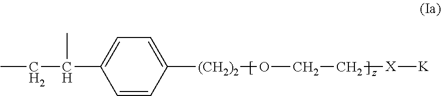
where
z is from 3 to 20;
X is a direct bond, oxygen, sulfur, —N(R¹)—, —C(═O)O—, —O(O═)C—, —N(R¹)(O═)C—, —C(═O)N(R¹)—, —O—CHR¹—O—, —OC(═O)N(R¹)—, —N(R¹)C(═O)O—, >C(═O) or >C(═S);
R¹ is hydrogen or C₁-C₄-alkyl; and
K is a transition metal complex.

2. The supported transition metal complex as claimed in claim 1, characterized in that z=10.

3. A supported transition metal complex based on a silica gel matrix comprising structural units of the formula (II): 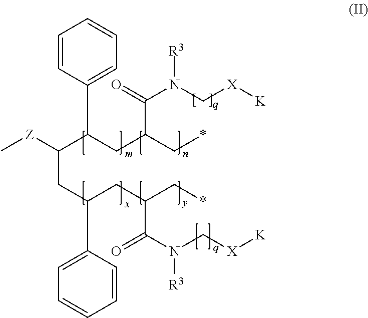
where
Z is a bond or a spacer;
m is from 1 to 5000;
n is from 1 to 5000;
x is from 1 to 5000;
y is from 1 to 5000;
X is a direct bond, oxygen, sulfur, —N(R¹)—, —C(═O)O—, —O(O═)C—, —N(R¹)(O═)C—, —C(═O)N(R¹)—, —O—CHR¹—O—, —OC(═O)N(R¹)—, —N(R¹)C(═O)O—, >C(═O) or >C(═S);
R¹ is hydrogen or C₁-C₄-alkyl;
R³ is hydrogen or C₁-Cₐ-alkyl;
q is from 2 to 5,
u is from 2 to 5 and
K is a transition metal complex.

4. The supported transition metal complex as claimed in claim 3, characterized in that q=3.

5. The supported transition metal complex as claimed in claim 3, characterized in that u=3.

6. The supported transition metal complex as claimed in claim 3 characterized in that R² and R³ are each methyl.

7. The supported transition metal complex as claimed in claim 1, characterized in that X is an amide bond, in particular —N(H)(O═)C—.

8. The supported transition metal complex as claimed in claim 1, characterized in that the transition metal complex is a molybdenum- or ruthenium-alkylidene complex.

9. The supported transition metal complex as claimed in claim 8, characterized in that the molybdenum- or ruthenium-alkylidene complex is derived from one of the following structures:
a structure of the formula (III): 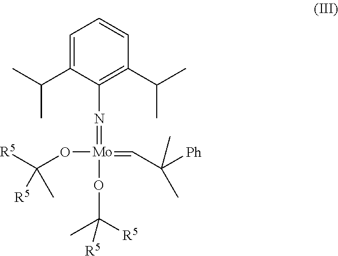
where
R⁵ is methyl or trifluoromethyl, or
a structure of the formula (IVa): 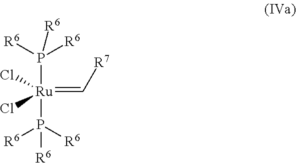
where
R⁶ is phenyl or cyclohexyl and
R⁷ is phenyl or CH═CPh₂, or
a structure of the formula (IVb): 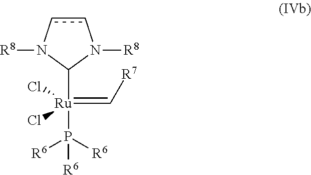
where
R⁶ is cyclohexyl;
R⁷ is phenyl; and
R⁸ is 2,4,6-trimethylphenyl (mesityl),
where “---” indicates an optional double bond, or
a structure of the formula (IVc): 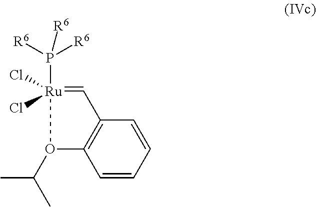
where
R⁶ is cyclohexyl, or
a structure of the formula (IVd): 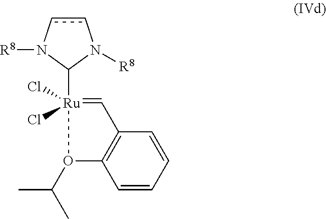
where
R⁸ is 2,4,6-trimethylphenyl (mesityl),
where “---” indicates an optional double bond.

10. The supported transition metal complex as claimed in claim 1, characterized in that K is a group of the formula (V): 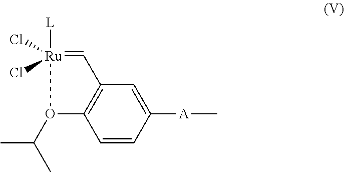
where
L is P(R⁶)₃ or 1,3-substituted 4,5-dihydroimidazol-2-ylidene, where R⁶ is cyclohexyl; and
A is C₁-C₁₀-alkylene or C₂-C₁₀-alkenylene bound to X.

11. (canceled)

12. A process comprising converting a starting material into a product in supercritical carbon dioxide, with a catalyst that is a supported transition metal complex as claimed in claim 1.

13. The process as claimed in claim 12, which comprises olefin metathesis.

14. The process as claimed in claim 13, characterized in that the olefin metathesis is a ring-closing metathesis.

15. The process as claimed in claim 12, characterized in that the process is carried out continuously.

16. The supported transition metal complex as claimed in claim 4, characterized in that u=3.

17. The supported transition metal complex as claimed in claim 4, characterized in that R² and R³ are each methyl.

18. The process as claimed in claim 13, characterized in that the process is carried out continuously.

19. The process as claimed in claim 14, characterized in that the process is carried out continuously.
